(19) United States
(12) Patent Application Publication
(10) Pub. No.: US 2013/0139305 A1
(43) Pub. Date: Jun. 6, 2013

(54) STRAP FOR PROTECTIVE EYEWEAR

(71) Applicant: Pam Rao, Vienna, VA (US)

(72) Inventor: Pam Rao, Vienna, VA (US)

(73) Assignee: RAO HOLDINGS, LLC, Vienna, VA (US)

(21) Appl. No.: 13/482,918

(22) Filed: May 29, 2012

Related U.S. Application Data
(60) Provisional application No. 61/575,186, filed on Aug. 17, 2011.

(51) Int. Cl. A61F 9/02

(52) U.S. Cl. CPC A61F 9/027
USPC 2/452

(57) ABSTRACT

An apparatus for maintaining protective eyewear, such as swimming goggles, on a wearer's head includes a neoprene backing and one or more nylon straps. The neoprene backing has a textured surface on the side that rests against a wearer's head. Hook and loop material attached to each end of the one or more nylon straps can be threaded through slots on each side of the protective eyewear to form an adjustable attachment of the strap to the protective eyewear.

### RELATED APPLICATIONS

The present application claims priority to U.S. Provisional Patent Application No. 61/575,186 filed on Aug. 17, 2011 by the same inventor, which Provisional Application is hereby incorporated by reference as if repeated herein in its entirety, including the drawings.

### BACKGROUND

The present invention relates generally to straps for use in wearing protective eye wear, and more particularly to a strap for wearing protective eye wear while swimming and/or diving. Such straps can be part of an entire set of goggles or the like, or the straps may be a separate device that one can use to attach to any pair of eye wear.

### Description of the Prior Art

Traditional swimming goggles are held in place on a swimmer's head using rubber straps. But these rubber straps get tangled in the swimmer's hair and are not easily adjusted while being worn. Typically, a swimmer must adjust the goggle strap, put the goggles on, test the goggles in the water, remove the goggles and readjust them to get the strap just right to prevent water from entering the eye socket of the goggles.

There are several patents that disclose swimming goggles equipped with an attachment that secures the goggles to a swimmer's head. When a rubber band is employed to attach the goggles to the head of a swimmer, the rubber band becomes easily dislodged, resulting in a poor fit. Moreover, the rubber band applies excessive pressure to the head of the swimmer, creating an unpleasant feeling in the wearer. Furthermore, the rubber bands do snap and break easily, especially after repeated wear in the chlorine or salt water and sunshine.

For example, U.S. Pat. No. 3,755,819 to Douglas discloses a swimming headgear, which is attached to the head of the user by a flexible elastic band. However, because the elastic band can be easily stretched, the headgear can become unsecure during active use. Moreover, this device suffers from the same problem as all elastic or rubber band related devices, i.e., that elastic or rubber bands get easily tangled with one's hair and are uncomfortable to the wearer. Furthermore, the rubber band is difficult to adjust.

As another example, U.S. Pat. No. 4,176,410 to Matthias discloses a sports goggle, which is attached to the head of the user using flexible straps that are adhesively attached to the outer ends of the sports goggle. These straps are made of a fabric with interwoven elastic filaments that permit elastic longitudinal elongation, which can lead to the device becoming dislodged during active swimming.

As an exemplary alternative to rubber or elastic bands, U.S. Pat. No. 4,243,041 to Paul discloses a set of goggles in which the goggles are secured around the head of a person using two separate detachable Velcro® strips, the first strip being attached by cementation to slots in the goggles frame. One of the strips carries the hook portion of Velcro® while the other strip carries the loop portion of the Velcro®. This attachment mechanism can fail to remain in place on a wearer during active use due to the location of the attachment means.

U.S. Pat. No. 5,046,200 to Feder discloses a reversible, quick-adjustable diver's face mask strap. A reversible, quick-adjustable diver's face mask strap includes a body portion formed of a flexible, elastic material. The strap further includes two ribbons attached to the body portion. Moreover, the double straps behind the head can easily become tangled with the wearer's hair. The split strap is more difficult to put on, especially for younger children.

U.S. Design Pat. D451,531 to Mabuchi discloses a pair of swimming goggles having an ornamental design. A foam rubber strap with a Velcro attachment on each of its ends is inserted into slots present in each side of a pair of swimming goggles. Since the rubber foam strap is expandable, the attachment of the swimming goggles to the swimmer is not tight and, due to the elasticity of the foam rubber strap, the goggle may migrate from one position to another during swimming. Moreover, the use of multiple straps in the rear leads to hair entanglement issues. Furthermore, foam and rubber break down from continued use in chlorinated or salt water and sunshine.

Notwithstanding the efforts of prior art workers, swimming goggles which are retained on the head of a swimmer without displacement during vigorous competitive swimming or conventional relaxed swimming to thereby prevent entry of water adjacent to the eyes of the swimmer are as yet not available. These rubberized straps must be tightened with excessive tension, causing discomfort to the swimmer.

U.S. Patent Application Publication No. 2008/0109949 A1 by Kinsella describes a swim goggles fastener system. However, this device gets tangled in one's hair and is not easily adjusted while already in place on a swimmer's head.

There accordingly remains a need in the art for swimming goggles that are easily attached and adjusted, and resist displacement, and remain securely in place during vigorous competitive swimming or relaxed swimming activities. Also needed are swimming goggles that exert minimal tension on the head of the swimmer, thereby providing a comfortable fit.

The present invention is therefore directed to the problem of developing an apparatus for use while swimming for maintaining protective eyewear comfortably in place that avoids the aforementioned problems.

### SUMMARY OF THE INVENTION

The present invention solves these and other problems by providing a strap for holding swimming goggles in place on a swimmer's head, which strap employs a large neoprene oval backing with a textured surface that fits around one's head in combination with nylon straps and hook and loop type fasteners (e.g., Velcro®) to attach the straps to the goggles through slots provided in the goggles.

The novel combination of large neoprene backing with a textured surface and thin nylon straps prevents the strap from tangling with the wearer's hair yet enables the goggles to be easily adjusted while being worn.

The neoprene backing has a wide width that enables attachment forces to be spread across the entire back of the wearer's head, thereby making the strap of the present invention comfortable to wear yet capable of applying sufficient force to maintain the strap in place on the wearer's head. By providing a large surface area upon which to contact the wearer, the total force exerted on the wearer can be larger without creating discomfort because only a small amount of force is felt at any one place. Additionally, the large neoprene backing increases the buoyancy making the goggles float, which is advantageous for swimming applications.

The textured surface creates a lower coefficient of friction with the wearer's hair, thereby improving the comfort while preventing entanglement issues.

Additionally, the present invention uses a hook and loop fastener type material (e.g., Velcro®) to enable the straps to be easily threaded through slots on the eye piece, while allowing for simply adjustment even while the goggles are already in place on the swimmer's head.

The length of the hook and loop fastener type material is quite long to enable the attachment to the goggles to remain in place even during rather energetic swimming. In an exemplary embodiment of the present invention, the length of the hook and loop fastener type material is about 3.5 inches on each side. One side of the strap uses the hook material sewn to the nylon, whereas the other side of the strap uses the loop material sewn to the nylon.

After the end of the strap is fed through the slot on the goggles, the hoop material is pressed against the loop material creating a strong attachment due to the area of the attachment, which can be approximately 9 cm by about 0.75 cm. Other sizes may suffice, and these values are merely examples.

Additionally, one exemplary embodiment of the present invention employs a wide oval shaped backing about 6 cm in width at the widest and tapering down from the maximum to a point where the straps are attached. The single wide backing provides a comfortable yet snug attachment to the wearer's head.

### BRIEF DESCRIPTION OF THE DRAWINGS

FIG. 1 depicts an exemplary embodiment of the present invention.

FIG. 2 shows the exemplary embodiment of FIG. 1 with one of the thin straps in an unmated position.

FIG. 3 depicts a top view of an exemplary embodiment of the present invention showing one side.

FIG. 4 depicts a top view of an exemplary embodiment of the present invention showing the other side from the side in FIG. 3, in which a textured surface on the backing can be seen.

FIG. 5 depicts a swimmer wearing an exemplary embodiment of the present invention.

### DETAILED DESCRIPTION

The present invention provides a colorful and stylish, yet comfortable neoprene and nylon swim goggle strap that replaces the rubber or bungee straps used in swimming goggles heretofore. An exemplary embodiment of the present invention employs an elliptically shaped neoprene backing in combination with two thin nylon straps, which have attached hook and loop fastener type material (e.g., Velcro®) at their ends. The end of the straps feeds through slots provided in eye wear, such as goggles, fold back against itself and forms an attachment when pressed against the opposite mating section of the hook and loop material. The combination of the neoprene backing and nylon strap prevents the swim goggle strap from tangling with one's hair.

Moreover, the novel combination also enables one to easily and comfortably adjust the swimming goggles while being worn. Previously, wearers of swimming goggles needed to remove the swim goggles to adjust the straps and then place the goggles back on their heads.

Another advantageous result of the combination of novel features of the present invention is that the swimming goggles of the present invention float. Losing one's swimming goggles causes one to cut short their swimming experience, but by floating the swimming goggles can be easily found should the goggles be inadvertently dropped in the water.

Yet another advantageous result of the novel combination is that the swimming goggles of the present invention can be easily marked with one's name or initials—preventing confusion when several people are using similar goggles. This identification helps prevent loss and theft of swim goggles.

The use of the neoprene backing and nylon strap enables the application of graphic designs and colors, thereby changing swimming goggle straps from being purely functional to also being fashionable and trendy.

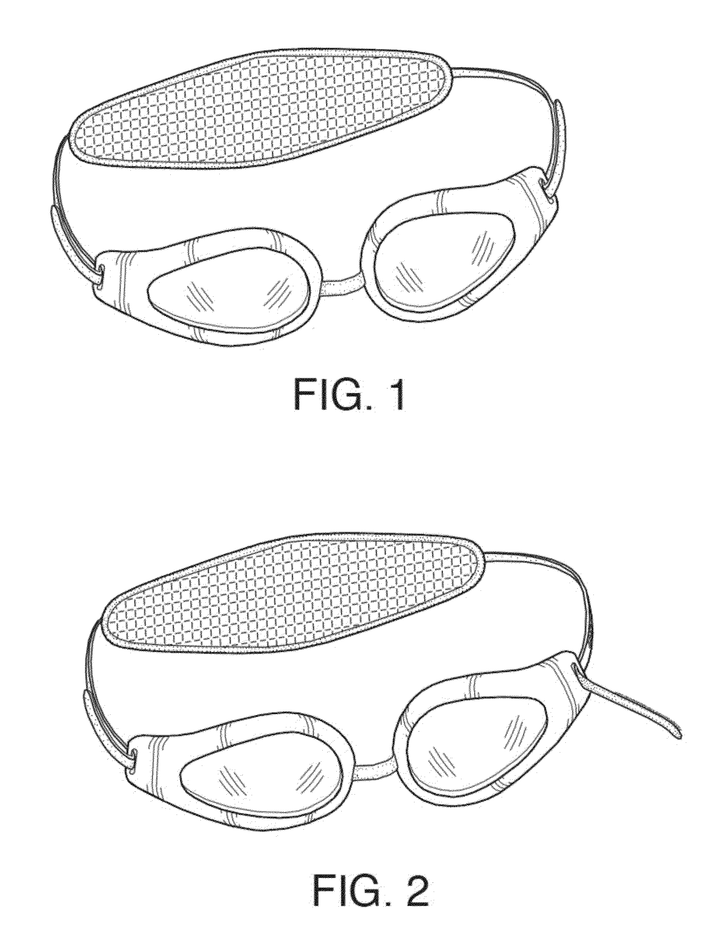

Turning to FIG. 1, shown therein is an exemplary embodiment 10 of the present invention. According to one aspect of the present invention, an exemplary embodiment 10 of a device for holding swimming goggles in place is constructed of two thin nylon side straps 13, 15 attached to a wide backing 12.

The wide backing 12 has an inside lining made of a non-slip material, such as rubber, textured neoprene or silicon, which prevents the neoprene strap from sliding down the back of the swimmers head. The neoprene can be made of a mid-grade quality neoprene, such as SBR or SCR. A higher quality of neoprene may suffice, such as CR, which is considered the best neoprene and often used for wetsuits.

The inside lining of the wide neoprene backing can have a textured surface to prevent the backing from becoming tangled in the swimmer's hair. The textured surface pattern is made by a machine mold. The opposite side is then covered in colorful fabric, which is sewn around the neoprene material.

One exemplary embodiment of a textured surface has ridges formed in each direction in a cross stitch pattern. Other embodiments could include bumps or indentations.

FIG. 2 shows the exemplary embodiment of FIG. 1 with one of the thin straps in an unmated position. This figure shows how the ends of the straps can be fed through slots on the goggles and then pressed against themselves to form a secure attachment, thereby holding the straps in place.

The thin straps of the present invention use one type of hook and loop material on one end of the straps and the opposite type of hook and loop material on the middle of the strap so that when the end of the strap is fed through the slot on the goggles and then folded back against itself the hook and loop material forms a strong bond. The inventor has found that using hook and loop material having an area of 9 cm by 1 cm forms a sufficient bond to maintain the straps in place on the goggles during vigorous swimming or diving. The hook and loop material can be sewn in place on the thin nylon straps.

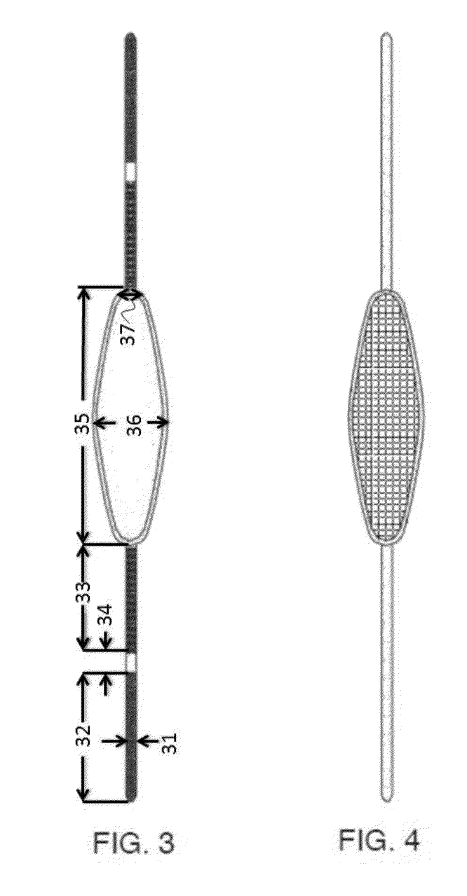

Turning to FIG. 3, shown therein is an exemplary embodiment of the strap of the present invention showing the various dimensions of the strap. Element 31 is the width of the strap, which is approximately 1 cm. The length 32 of the hook and loop material is approximately 9 cm. The section 34 is the portion of the strap that folds around the slot for the goggles so that section 32 mates with section 33 when pressed together. The mating section is therefore about 9 cm in length. Section 34 is about 2.5 cm in length. The width of the oval neoprene section is about 6 cm at the widest point 36. The length 35 of the oval neoprene section is about 18 cm in length. The thickness of the neoprene backing is about 4 mm. There is a 0.7 cm edging around the neoprene backing so the textured surface area is about 16.6 cm long and about 4.6 cm wide. The inventor has found that these dimensions serve to enable the wearer to feel comfortable when swimming with the goggles and strap of the present invention in place on the swimmer's head, yet can be easily adjusted.

While these dimensions are provided as an exemplary embodiment, the present invention is not limited to these dimensions. Moreover, the thinner side straps helps to prevent tangling with the wearer's hair. The single solid piece backing is also meant for minimizing tangling and ease of putting on and taking off. The entire design is meant for easy on and easy off with minimum tangling (as well as ease of adjustment while on). The one piece back and thin side straps helps increase buoyancy making the goggles/strap float, which is advantageous to swimmers. The one piece strap makes it easier for younger users to don the product themselves. Last but maybe not least, the thinner side straps make the strap more universal to a variety of sizes and styles of swim goggle frames.

According to one aspect of the present invention, an exemplary embodiment of a strap for swimming goggles uses hook and loop material 11, 14 affixed to the nylon side straps 13, 15, respectively, which hook and loop material 11, 14 is threaded through side openings of the swimming goggles (not shown), and can then be secured using the hook and loop material. This enables quick adjustment while the swimmer is wearing the goggles.

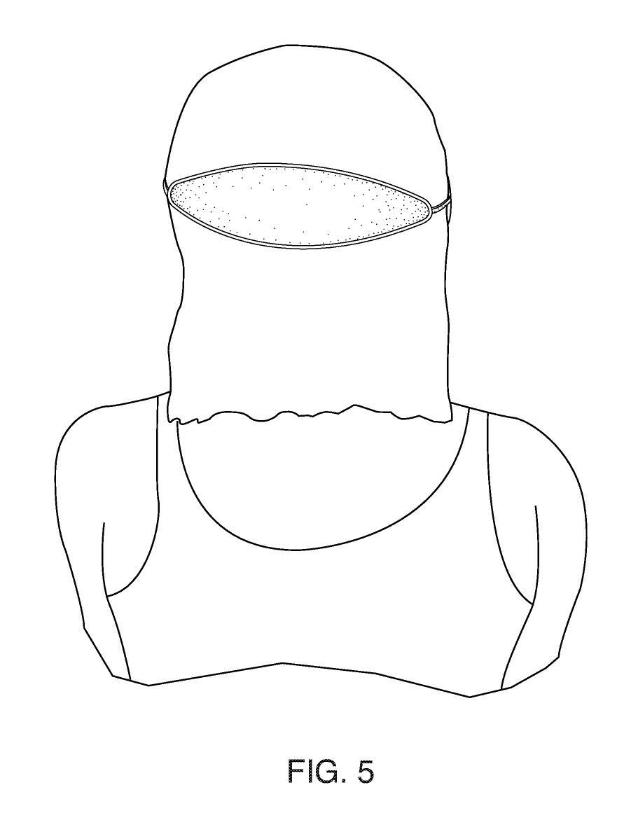

FIG. 4 depicts the opposite side of the embodiment of FIG. 3 in a top view.

Shown in FIG. 5 is the exemplary embodiment of the present invention as worn by a swimmer.

What is claimed is:

1. An apparatus for maintaining protective eyewear on a wearer's head, said protective eyewear having a slot on each side, said apparatus comprising:
at least one nylon strap having two sides and including a first end being of a width sufficient to pass through one of the slots on the protective eyewear, said first end having a first hook-and-loop type fastener material attached on a first one of the two sides, said at least one nylon strap having a first middle area on said first one of the two sides in which a second hook-and-loop type fastener material is attached, wherein said first nylon strap can be passed through said one of the slots and folded back on itself so that the first hook-and-loop type fastener material can be pressed against the second hook-and-loop type material to form a first adjustable attachment; and
a neoprene backing attached to the at least one nylon strap, said neoprene backing having a width being substantially wider than a width of the at least one nylon strap, said neoprene backing having a length approximately a length of the protective eyewear, said neoprene backing having a textured surface on at least one side to rest against a wearer's head.

2. The apparatus of claim 1, further comprising:
a second nylon strap having two sides and including a first end being of a width sufficient to pass through another one of the slots on the protective eyewear, said first end of the second nylon strap having a third hook-and-loop type fastener material attached on a first one of the two sides of the second nylon strap, said second nylon strap having a second middle area on said first one of the two sides of the second nylon strap in which a fourth hook-and-loop type fastener material is attached, wherein said second nylon strap can be passed through said another one of the slots and folded back on itself so that the third hook-and-loop type fastener material can be pressed against the fourth hook-and-loop type material to form a second adjustable attachment.

3. The apparatus according to claim 1, wherein the neoprene backing has an oval shape.

4. The apparatus according to claim 1, wherein the first hook and loop type fastener material has a surface area of about 9 cm by 1 cm.

5. The apparatus according to claim 1, wherein the second hook and loop type fastener material has a surface area of about 9 cm by 1 cm.

6. The apparatus according to claim 1, wherein the neoprene backing has an oval shape about 18 cm in length.

7. The apparatus according to claim 1, wherein the at least one strap is about 1 cm in width.

8. The apparatus according to claim 2, wherein the second strap is about 1 cm in width.

9. The apparatus according to claim 1, wherein the neoprene backing has an oval shape about 5 cm in width.

10. The apparatus according to claim 1, wherein the neoprene backing has an oval shape about 18 cm in length and about 5 cm in width.

11. An apparatus for protecting eyes of a wearer comprising:
a first protective goggle to protect one eye of the wearer;
a second protective goggle to protect another eye of the wearer;
a thin member coupling the first protective goggle to the second protective goggle;
a first slot disposed in the first protective goggle having a width;
a second slot disposed in the second protective goggle having a width;
at least one nylon strap having two sides and including a first end being of a width sufficient to pass through the first slot, said first end having a first hook-and-loop type fastener material attached on a first one of the two sides, said at least one nylon strap having a first middle area on said first one of the two sides in which a second hook-and-loop type fastener material is attached, wherein said at least one nylon strap can be passed through said first slot and folded back on itself so that the first hook-and-loop type fastener material can be pressed against the second hook-and-loop type material to form a first adjustable attachment; and
a neoprene pad attached to the first and second nylon straps, said neoprene pad having a width being substantially wider than a width of the at least one nylon strap, said neoprene pad having a length approximately a distance from one end of the first protective goggle to an end of the second protective goggle when in place on the wearer, said neoprene backing having a textured surface on at least one side to rest against the wearer's head.

12. The apparatus according to claim 11, further comprising:
a second nylon strap having two sides and including a first end being of a width sufficient to pass through the second slot, said first end of the second nylon strap having a third hook-and-loop type fastener material attached on a first one of the two sides of the second nylon strap, said second nylon strap having a second middle area on said first one of the two sides of the second nylon strap in which a fourth hook-and-loop type fastener material is attached, wherein said second nylon strap can be passed through said second slot and folded back on itself so that the third hook-and-loop type fastener material can be pressed against the fourth hook-and-loop type material to form a second adjustable attachment.

13. The apparatus according to claim 11, wherein the neoprene pad has an oval shape.

14. The apparatus according to claim 11, wherein the first hook and loop type fastener material has a surface area of about 9 cm by 1 cm.

15. The apparatus according to claim 11, wherein the second hook and loop type fastener material has a surface area of about 9 cm by 1 cm.

16. The apparatus according to claim 11, wherein the neoprene pad has an oval shape about 18 cm in length.

17. The apparatus according to claim 11, wherein the at least one strap is about 1 cm in width.

18. The apparatus according to claim 12, wherein the second strap is about 1 cm in width.

19. The apparatus according to claim 11, wherein the neoprene pad has an oval shape about 5 cm in width.

20. The apparatus according to claim 11, wherein the neoprene pad has an oval shape about 5 cm in width and 18 cm in length.
